(19) United States
(12) Patent Application Publication
(10) Pub. No.: US 2006/0199849 A1
(43) Pub. Date: Sep. 7, 2006

(54) Solid lercanidipine free base

(75) Inventors: Fabio Berlati, Milan (IT); Amedeo Leonardi, Milan (IT); Gianni Motta, Barlassina (IT); Ilaria Candiani, Busto Arsizio (IT); Francesco Corcella, Parabiago (IT)

Correspondence Address: DARBY & DARBY P.C., P. O. BOX 5257, NEW YORK, NY, 10150-5257, US

(73) Assignee: Recordati Ireland Limited, Ringaskiddy (IE)

(21) Appl. No.: 11/364,861

(22) Filed: Feb. 27, 2006

Related U.S. Application Data
(60) Provisional application No. 60/656,741, filed on Feb. 25, 2005.

(51) Int. Cl. C07D 211/82; A61K 31/44

(52) U.S. Cl. 514/355; 546/315

(57) ABSTRACT

The invention provides substantially pure lercanidipine free base, having a purity of at least 95%, preferably at least 97%, more preferably at least 99%, and still more preferably at least 99.5%. The lercanidipine free base of the present invention is formed as an amorphous solid that is easily handled and particularly well suited to the formulation of pharmaceutical compositions.

This application claims the benefit of U.S. Provisional Patent Application No. 60/656,741, filed Feb. 25, 2005, which is hereby incorporated by reference in its entirety.

### FIELD OF THE INVENTION

The invention relates to substantially pure lercanidipine free base, to methods of preparing the free base, and to pharmaceutical compositions comprising the same.

### BACKGROUND OF THE INVENTION

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist with a long duration of action and high vascular selectivity. Lercanidipine's biological activity derives from its ability to competitively antagonize the dihydropyridine subunit of the L-type calcium channel.

Lercanidipine is useful as an anti-hypertensive. Lercanidipine lowers blood pressure by blocking calcium channels of arterial smooth muscle, thus decreasing peripheral vascular resistance. Lercanidipine produces no negative cardiac inotropism and only occasional mild reflex tachycardia, which is generally of short duration. Lercanidipine has been approved for the treatment of hypertension and has been marketed since 1996 in several European countries under the trademark Zanidip™.

The hydrochloride salt of lercanidipine is commercially available from Recordati S.p.A. (Milan, Italy). Methods of preparing lercanidipine hydrochloride, as well as methods of resolving lercanidipine into individual enantiomers are described in U.S. Pat. Nos. 4,705,797; 5,767,136; 4,968,832; 5,912,351; and 5,696,139, 6,852,737 and U.S. application, Publication No. 2003/0083355, all of which are incorporated herein by reference.

A method of preparing crude lercanidipine free base is disclosed in U.S. Pat. No. 4,705,797. In the method, a solution of methyl 3-aminocrotonate and 1,1,N-trimethyl-N-(3,3-diphenylpropyl)-2-aminoethyl α-acetyl-3-nitrocinnamate was cyclized, followed by drying. The product was purified by flash chromatography on silica gel columns, using chloroform with increasing amounts of acetone as eluent. The resulting crude lercanidipine free base is an amorphous, low melting solid composition having a purity of approximately 94%, as measured by high pressure liquid chromatography (HPLC).

The amorphous lercanidipine free base of the prior art is not well suited for formulation into pharmaceutical compositions because it is impure. Pharmaceutical compositions require that the active agent be substantially pure, i.e., at least 99% pure. The amorphous lercanidipine of the prior art however, is only about 94% pure. Preparation of a pharmaceutically suitable free base using the prior art method of preparation and purification is commercially impracticable because it is expensive, time consuming and results in relatively low yields of product. Additionally, the free base of the prior art is viscous making it difficult to handle during the formulation process and therefore is not well suited for use in pharmaceutical compositions and solid dosage forms of the prior art, i.e. immediate release tablets. For these reasons, the free base of the prior art and the method of preparing the same are poorly suited for the preparation of pharmaceutical compositions.

Only recently has it been discovered that amorphous compositions, and in particular amorphous lercanidipine, are well suited for use in modified release capsules comprising waxy substances. Therefore, to facilitate the development of amorphous lercanidipine pharmaceutical compositions, there is a need in the art for a substantially pure amorphous lercanidipine free base and a method of producing the same, that overcomes the limitations of prior art. Preferably, the method yields an amorphous lercanidipine free base that is substantially pure, easily handled and easily incorporated into pharmaceutical compositions and oral dosage forms. Additionally, it is preferred that the resulting amorphous lercanidipine free base have similar or improved characteristics, e.g., solubility and bioavailability, compared to lercanidipine of the prior art.

Accordingly, the present inventors have discovered a substantially pure lercanidipine free base having significantly greater purity than free base prepared by the prior art method. The novel method for preparing lercanidipine free base is rapid, simple, yields a substantially pure product, and is well suited for commercialization. The lercanidipine free base of the present invention has greater solubility in organic solvents than lercanidipine hydrochloride, and unexpectedly improved bioavailability when administered to a mammal. Additionally, the presently described lercanidipine free base is easily formulated in oral dosage forms and is particularly well suited for incorporation into modified release pharmaceutical dosage forms comprising waxy substances as, e.g., a diffusion matrix. Furthermore, modified release compositions comprising substantially pure lercanidipine free base will demonstrate greater bioavailability compared to commercially available compositions comprising crystalline lercanidipine hydrochloride.

### SUMMARY OF THE INVENTION

The present invention is directed to a substantially pure lercanidipine free base, as well as to methods for making it and compositions comprising it. The lercanidipine free base of the present invention is prepared as a substantially amorphous solid, having purity greater than that of crude lercanidipine free base of the prior art. In one embodiment, the present invention provides for a lercanidipine free base, having a purity of at least 95% and preferably at least about 97%, more preferably at least about 99% and still more preferably at least about 99.5%.

In another aspect, the present invention provides a method of preparing substantially pure lercanidipine free base having a purity of at least 95% by alkalization of a lercanidipine salt. In one embodiment, substantially pure free base is prepared by (a) dissolving a lercanidipine salt in an organic solvent to form a solution, (b) combining the solution and an aqueous medium having a pH in the range from about 9 to about 14; and (c) isolating the substantially pure lercanidipine free base. Preferably the lercanidipine salt is at least 95% pure.

In another embodiment, substantially pure free base is prepared by (a) suspending a lercanidipine salt in an water immiscible organic solvent and water, (b) adding to this stirred mixture an inorganic solid base and continuing stirring, (c) isolating the organic phase and evaporating the solvent under vacuum, (d) dissolving the so obtained pure lercanidipine free base in a miscible organic solvent and combining this solution with water, and (e) isolating the substantially pure lercanidipine free base.

In still another aspect, the present invention provides for a pharmaceutical composition comprising, (a) substantially pure lercanidipine free base; and (b) a pharmaceutically acceptable excipient and/or carrier.

In yet other aspects, the present invention provides a modified release pharmaceutical composition comprising at least one polyglycolized glyceride and a therapeutically effective amount of substantially pure lercanidipine free base dispersed in said polyglycolized glyceride, wherein the polyglycolized glyceride has a melting point from about 40° C. to about 60° C. and a hydrophobic lipophilic balance (HLB) value from about 1 to about 14.

In a preferred embodiment, the modified release pharmaceutical composition of the present invention comprises at least one polyglycolized glyceride and a therapeutically effective amount of substantially pure lercanidipine free base, wherein the polyglycolized glyceride is selected from the group consisting of Gelucire™ 37/02, 37/06, 42/12, 44/14, 46/07, 48/09, 50/02, 50/13, 33/01, 39/01, 43/01, and 53/10, or a combination thereof.

In still other aspects, the present invention provides for solid dosage forms comprising the pharmaceutical compositions disclosed herein.

### DETAILED DESCRIPTION OF THE INVENTION

As used herein, the following terms are defined as follows:

The term “about” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the purity of the compositions of the present invention can mean a range of preferably ±0.5%, more preferably ±0.25% and still more preferably ±0.1% of a particular value.

The term “substantially pure” refers to a composition that is at least 95% pure, preferably at least at least about 97% pure, and more preferably at least about 99% pure on weight/weight basis relative to contaminants, including solvents carried over from the preparation of the composition.

The term “lercanidipine free base” refers to methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate and excludes other forms of the active moiety, e.g., acid addition salts.

The term “crude” refers to a composition that is less than 94% pure on weight/weight basis relative to contaminants, including solvents carried over from the preparation of the composition.

The term “lercanidipine salt” refers to a salt of lercanidipine. Acceptable salts include, but are not limited to lercanidipine salts formed with inorganic or organic acids, such as (i) inorganic acids, such as hydrochloric acid, hydrobromic acid, phosphoric acid and sulphuric acid; (ii) sulphuric acids, such as methanesulphuric acid, benzenesulphuric acid, toluenesulphuric acid, and napthalene-1,5-disulphuric acid, (iii) monocarboxylic acids, such as acetic acid, (+)-L-lactic acid, DL-lactic acid, DL-mandelic acid, gluconic acid, cinnamic acid, salicylic acid, and gentisic acid, (iv) dicarboxylic acids, such as oxalic acid, 2-oxo-glutaric acid, malonic acid, (−)-L-malic acid, mucic acid, (+)-L-tartaric acid, fumaric acid, maleic acid, and terephthalic acid, (v) tricarboxylic acids, such as citric acid, and (vi) aromatic sulphonimides such as saccharin. Preferred pharmaceutically acceptable salts of lercanidipine, include but are not limited to, the hydrochloride, besylate and napadisylate salts. The lercanidipine salt may be present in one or more crystalline or amorphous forms.

The term “amorphous” refers to a solid compound having no substantial crystal lattice structure. In one preferred embodiment, amorphous compounds are identified by DSC analysis. Typically, amorphous compounds have DSC plots with broad endothermic transitions, defined as glass transition, rather then sharp exothermic peaks typical of crystalline compounds. Additionally, amorphous compounds present XRD spectra having broad shoulders rather than well-defined peaks profile, which are characteristic of the crystalline solids.

The term “modified release” refers to release of the active ingredient, lercanidipine, from a composition of the present invention over a period of time sufficient to maintain therapeutically effective plasma levels over a similarly extended time interval and/or to modify other pharmacokinetic properties of the active ingredient. Preferably modified release results in therapeutic plasma concentrations of lercanidipine for a period of about 20 to about 25 hours and a mean plasma concentration of lercanidipine of greater than 0.5 ng/mL over the duration of the dosing interval.

The term “pharmaceutically acceptable” refers to a material, device or process that is biologically or pharmacologically compatible for in vivo use. In one aspect the term characterizes those substances that are approved by a regulatory agency of the U.S., or a state government or listed in the U.S. Pharmacopoeia or other generally recognized pharmacopoeia for use in animals, and more particularly in humans.

The term “therapeutically effective amount” refers to the amount of active agent sufficient to lower the blood pressure of a patient with hypertension. Therapeutically effective amounts of active agent preferably lower blood pressure, such that the values for systolic and diastolic blood pressure are below 140 and 90 mm Hg, respectively. A therapeutically effective amount of the active agent may or may not decrease the blood pressure in a person that does not have hypertension or may not decrease blood pressure in all persons with hypertension. Therapeutic effectiveness in treatment of other pathologies, such as heart failure or atherosclerosis is also specifically contemplated as per, e.g., U.S. Pat. Nos. 5,696,139 and 5,767,136, which are incorporated herein by reference. Preferably, a therapeutically effective amount of active agent leads to a reduction in blood pressure, e.g., within about 2 to 6 hours. Preferably, when a rapid reduction in blood pressure is desired, a therapeutically effective amount of active agent will reduce systolic blood pressure in the range from about 20-30 mm Hg and diastolic blood pressure in the range from about 10-20 mm Hg, within about 30 minutes to about 60 minutes following administration of the active agent.

The term “patient” refers to a mammal (e.g., a human) suffering from or at risk of developing the particular condition to be treated, e.g., essential hypertension, secondary hypertension, isolated systolic hypertension, coronary heart disease (e.g., chronic stable angina, myocardial infarction), congestive heart failure, hypertensive chrysis or angina. A patient in need of treatment for arterial hypertension may be identified using methods well-known in the art, for example by direct measurement of blood pressure using a manual sphygmomanometer, automatic/electronic devices or ambulatory blood pressure monitoring.

Preparation of Lercanidipine Free Base

The present invention provides a substantially pure lercanidipine free base, particularly an amorphous lercanidipine free base having a purity of at least about 95% and more preferably at least about 97% and still more preferably at least about 99%. The purity of the free base may be determined by any method known in the art, including, but not limited to high performance liquid chromatography (HPLC) analysis.

The lercanidipine free base of the present invention, is easily obtained in a highly purified state, practically free from contaminants. The inventors have discovered that the substantially pure lercanidipine free base has physical properties, e.g., solubility and bioavailability, that are preferable to physical properties of other known forms of lercanidipine. The substantially pure free base of the present invention is more easily handled compared to crude lercanidipine free base prepared by the method of the prior art and may be readily incorporated into pharmaceutical compositions and solid dosage forms.

In one embodiment, the lercanidipine free base of the present invention may be prepared by alkalization of a lercanidipine salt in the presence of an organic solvent. The lercanidipine salt may be any salt known in the art, including, but not limited to, the hydrochloride, besylate and napadisylate salts. The lercanidipine salts may be prepared using methods well known in the art, such as those disclosed in U.S. patent application Ser. No. 11/211,769, which is incorporated herein by reference, or from commercial sources. One particularly preferred lercanidipine salt is lercanidipine hydrochloride.

Alkalization of a lercanidipine salt to yield the free base may be carried out by combining a lercanidipine salt dissolved in an organic solvent with an aqueous medium having a pH in the range from about 9 to about 14. The alkalization reaction may be carried out at temperature from about 0° C. to about 25° C., preferably at a temperature from about 5° C. to about 20° C. Preferably the reaction components are stirred upon combination for a period from about 30 to about 120 minutes, then allowed to stand for a period from about 1 to about 12 hours.

Preferably, the preparation of substantially pure lercanidipine free base from a lercanidipine salt is carried out in the presence of an organic solvent. In one embodiment, the lercanidipine salt or crude lercanidipine free base is dissolved in a miscible organic solvent prior to combining with a basic aqueous medium. Preferred miscible organic solvents include, but are not limited to protic polar or aprotic polar solvents. Protic polar solvents include, but are not limited to, for example, simple alcohols such as, methanol, ethanol, propanol, and polyalcohols, such as ethylene glycol. Typical aprotic polar solvents include, but are not limited to, for example N,N-dimethylformamide,dimethylsulphoxide, dimethylacetamide. One particularly preferred miscible solvent is methanol.

Preferably the preparation of the free base of the present invention is carried out in an aqueous medium having a pH from about 9 to about 14 more preferably from about 9.2 to about 10, and most preferably about 9.2. Preferred, aqueous media include, but are not limited to, aqueous media comprising organic and inorganic bases. Suitable organic bases include, but are not limited to, triethylamine, piperazine, tetramethylethylenediamine, ethylenediamine, 4-dimethylaminopyridine. Suitable inorganic bases include, but are not limited to, hydroxides like NaOH, KOH, LiOH and borax (Na₂B₄O₇.10H₂O) or basic salts such as sodium carbonate or potassium carbonates. Also an anionic ion exchange resin can be used. Preferred anion exchange resins are commercially available strong base ion exchange resins, including those containing strongly basic (cationic) groups such as quaternary ammonium groups, tertiary sulphonium groups, quaternary phosphonium groups or alkyl pyridinium groups. Particularly preferred anion exchange resins are those containing quaternary amines, such as Rexyn™ 201 (Fisher Scientific Co.), Amberlite™ IR A-400, (Mallinckrodt Chemical Works), Ionac™ A-540 (Matheson, Coleman and Bell), Dowex™ I and 21K (Dow Chemical Co.), and Duolite™ A-101D and ES-109 (Diamond Shamrock Chemical Co.). In instances when ion-exchange resins are used, the resin is filtered away from the organic solution prior to addition of water.

One particularly preferred aqueous medium for the alkalization step is a buffered solution having a pH of about 9.2, comprising borax (Na₂B₄O₇.10H₂O).

In another embodiment the organic solvent is immiscible with water. Examples of immiscible organic solvents include, but are not limited to, hydrocarbons, such as toluene, halogenated hydrocarbons, such as methylene chloride, esters, such as methyl acetate, ethyl acetate, and ethers, such as diethyl ether and methyl tert-butyl ether. One particularly preferred immiscible solvents is ethyl acetate. A lercanidipine salt is suspended in mixture of at least one immiscible organic solvent and water and added under stirring with at least one solid inorganic base such as potassium hydroxide, sodium hydroxide, sodium carbonate or potassium carbonate. One particularly preferred inorganic base is potassium carbonate.

After mixing, the aqueous phase is removed and the solvent is evaporated. The resulting free base is dissolved in a second organic solvent, preferably an organic solvent that is miscible in water, such as those discussed above. After the free base is dissolved in the second organic solvent, the free base is precipitated from the solution, preferably by the addition of water.

The substantially pure free base, resulting from alkalization of a lercanidipine salt, may be isolated using simple separation techniques well known in the art. The ease with which the lercanidipine free base of the present invention may be isolated is an additional advantage of the present discovery over the prior art method of preparing lercanidipine free base. Following alkalization, the free base may be isolated from the reaction mixture using any separation technique known in the art including, but not limited to vacuum filtration. Upon isolation from the reaction medium the free base may be dried using any drying technique known in the art.

The resulting lercanidipine free base is substantially pure, i.e., having a purity of at least 95% and more preferably at least about 97% and still more preferably at least about 99%. The lercanidipine free base of the present invention is formed as an amorphous solid that is easily handled and particularly well suited to the formulation of pharmaceutical compositions. The melting point of the lercanidipine free base is from 40° C. to 70° C., more specifically in the range from 44° C. to 64° C., when determined by open capillary method.

Pharmaceutical Compositions

The substantially pure lercanidipine free base of the present invention may be formulated into pharmaceutical compositions. A pharmaceutical composition according to the invention also may include one or more optional excipients or additives, such as a pharmaceutically acceptable carrier or diluent, a flavorant, a sweetener, a preservative, a dye, a binder, a suspending and/or viscosity-increasing agent, a dispersing agent, a colorant, a disintegrant, an excipient, a film forming agent, a lubricant, a plasticizer, an edible oil or any combination of two or more of the foregoing, an antioxidant, a chelating agent, a buffering agent, solubilizing agents, a wetting agent and a glidant and combinations of two or more of the foregoing.

Preferably, the pharmaceutical compositions of the present invention comprise a sufficient amount of substantially pure lercanidipine free base to render a therapeutic effect when the composition is administered to a patient. Lercanidipine free base may be present in any amount from about 0.001 to about 0.2 mg per mg of the total composition, and more preferably from about 0.002 mg to about 0.1 mg per mg of the total composition and most preferably 0.005 mg about 0.1 mg per mg of the total composition.

In other embodiments, the pharmaceutical composition of the present invention may comprise a mixture of a substantially pure lercanidipine free base and at least one lercandipine salt.

Suitable pharmaceutically acceptable carriers or diluents include, but are not limited to, ethanol; water; fatty acid glycerides, stearolyl macrogol glicerides, lauroyl macrogol glycerides; glycerol; propylene glycol, aloe vera gel; allantoin; glycerin; vitamin A and E oils; mineral oil; PPG2 myristyl propionate; magnesium carbonate; potassium phosphate; vegetable oil; animal oil; and solketal.

Suitable binders include, but are not limited to, starch; gelatin; natural sugars, such as glucose, sucrose and lactose; corn sweeteners; natural and synthetic gums, such as acacia, tragacanth, vegetable gum, and sodium alginate; carboxymethylcellulose; hydroxypropylmethylcellulose; polyethylene glycol; povidone; waxes; and the like. Preferred binders are lactose, hydroxypropylmethylcellulose and povidone.

Suitable disintegrants include, but are not limited to, starch (e.g., corn starch or modified starch) methyl cellulose, sodium crosscarmellose, agar, bentonite, xanthan gum, sodium starch glycolate, crosspovidone and the like. A preferred disintegrant is sodium starch glycolate.

Suitable lubricants include, but are not limited to, sodium oleate, sodium stearate, sodium stearyl fumarate, magnesium stearate, sodium benzoate, sodium acetate, sodium chloride and the like. A preferred lubricant is magnesium stearate.

A suitable suspending agent is, but is not limited to, bentonite, ethoxylated isostearyl alcohols, polyoxyethylene sorbitol and sorbitan esters, microcrystalline cellulose, aluminum metahydroxide, agar-agar and tragacanth, or mixtures of two or more of these substances, and the like. A preferred suspending agent is microcrystalline cellulose.

Suitable dispersing and suspending agents include, but are not limited to, synthetic and natural gums, such as vegetable gum, tragacanth, acacia, alginate, dextran, sodium carboxymethylcellulose, methylcellulose, polyvinyl-pyrrolidone and gelatin.

Suitable film forming agents include, but are not limited to, hydroxypropylmethylcellulose, ethylcellulose and polymethacrylates.

Suitable plasticizers include, but are not limited to, polyethylene glycols of different molecular weights (e.g., 200-8000 Da) and propylene glycol. Preferred is polyethylene glycol 6000.

Suitable colorants include, but are not limited to, ferric oxide(s), titanium dioxide and natural and synthetic lacquers. Preferred are ferric oxides and titanium dioxide.

Suitable edible oils include, but are not limited to, cottonseed oil, sesame oil, coconut oil and peanut oil.

Suitable antioxidants include, but are not limited to, butylated hydroxytoluene, butylated hydroxyanisole, ascorbyl palmitate, tocopherol, tocopheryl acetate, etc.

Examples of additional additives include, but are not limited to, sorbitol, talc, stearic acid, dicalcium phosphate and polydextrose.

In a preferred embodiment, the invention provides a modified release pharmaceutical composition comprising at least one fatty acid glycerides and a therapeutically effective amount of substantially pure lercanidipine free base.

Fatty acid glycerides suitable for use in modified release formulations include both medium chain and long chain fatty acid glycerides. In one aspect, the pharmaceutical compositions of the present invention may include one or more long chain (C12 to C22) fatty acid glycerides (including monoesters, diesters and/or triesters of glycerol). Examples of long chain fatty acid glycerides, within the scope of the present invention are Compritol 888 ATO™ and Precirol ATO 5 ™ (commercially available from Gattefossé Corporation, Paramus, N.J.).

Additional preferred fatty acid glycerides, suitable for use herein include one or more medium chain (C₈ to C₁₁) fatty acid glycerides such as one or more triglycerides of C₈ to C₁₁ fatty acids. One example of one medium chain fatty acid triglyceride, within the scope of the present invention is Miglyol™ 812 (commercially available from Condea Chemie GmbH, Cranford, N.J.).

Polyethylene glycol esters and polypropylene esters suitable for use in modified release formulations include mono- and diesters of polyethylene glycols and polypropylene glycols. Suitable and preferred fatty acids for inclusion in polyethylene glycol esters and polypropylene glycol esters are C₁₂ to C₂₂ fatty acids, as set forth above. Suitable polyethylene glycol chains and polypropylene chains for use respectively in polyethylene glycol esters and polypropylene glycol esters are described in, e.g., the U.S. Pharmacopeia.

Preferred fatty acid glycerides for use in the present modified release compositions, have a melting point from about 40° C. to about 80° C. and a HLB value from about 1 to about 14.

“Polyglycolized glycerides” denotes a mixture of mono-, di- and triglycerides and polyethylene glycol (PEG) mono- and diesters. Polyglycolized glycerides are particularly preferred waxy substances for use in the present invention. Polyglycolized glycerides are commercially available under the name Gelucire™ (Gattefossé Corporation, Paramus, N.J.).

Particular grades of Gelucire™ which are useful in the present invention, include, but are not limited to Gelucire™ 37/02, 37/06, 42/12, 44/14, 46/07, 48/09, 50/02, 50/13, 33/01, 39/01, 43/01 and 53/10, or combinations thereof. The first number in the nomenclature of a Gelucire™ denotes its melting point while the second number characterizes its HLB value. For example, Gelucire™ 50/13 has a melting point of about 55° C., and an HLB value of about 13. Particularly preferred grades of Gelucire™, are Gelucire™ 50/13, and Gelucire™ 44/14 or combinations thereof.

The pharmaceutical composition may optionally include additives, such as for example, pharmaceutically acceptable carriers or diluents, flavorants, sweeteners, preservatives, antioxidants, wetting agents, buffering agents, release controlling agents, dyes, binders, suspending agents, dispersing agents, colorants, disintegrants, excipients, film forming agents, lubricants, plasticizers, edible oils or any combination of two or more of the foregoing. The composition may be related to solid pharmaceutical forms as hard capsule and soft capsules, tablets, coated tablets, or sachets. Suitable pharmaceutically acceptable carriers or diluents include, but are not limited to, ethanol; water; glycerol; propylene glycol; glycerin; diethylene glycol monoethylether, vitamin A and E oils; mineral oil; PPG2 myristyl propionate; magnesium carbonate; potassium phosphate; silicon dioxide; vegetable oil; animal oil; and solketal.

Unit Dosage Forms

The pharmaceutical composition may be formulated as unit dosage forms, such as tablets, pills, capsules, caplets, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. Unit dosage forms may be used for oral, parenteral, intranasal, sublingual or rectal administration, or for administration by inhalation or insufflation, transdermal patches, and a lyophilized composition. In general, any delivery of active ingredients that results in systemic availability of them can be used. Preferably the unit dosage form is an oral dosage form, most preferably a solid oral dosage form, therefore the preferred dosage forms are tablets, pills, caplets and capsules. However, in an additional preferred embodiment, unit dosage forms may be provided as parenteral preparations.

In another embodiment, solid unit dosage forms may be prepared by mixing the lercanidipine free base of the present invention with a pharmaceutically acceptable carrier and any other desired additives as described above. The mixture is typically mixed until a homogeneous mixture of the active agents of the present invention and the carrier and any other desired additives is formed, i.e., until the active agents are dispersed evenly throughout the composition. In this case, the compositions can be formed as dry or moist granules.

For liquid dosage forms, the active substances or their physiologically acceptable salts are brought into solution, suspension or emulsion, optionally with the usually employed substances such as solubilizers, emulsifiers or other auxiliaries. Solvents for the active combinations and the corresponding physiologically acceptable salts, can include water, physiological salt solutions or alcohols, e.g. ethanol, propane-diol or glycerol. Additionally, sugar solutions such as glucose or mannitol solutions may be used. A mixture of the various solvents mentioned may further be used in the present invention.

A transdermal dosage form also is contemplated by the present invention. Transdermal forms may be a diffusion-driven transdermal system (transdermal patch) using either a fluid reservoir or a drug-in-adhesive matrix system. Other transdermal dosage forms include, but are not limited to, topical gels, lotions, ointments, transmucosal systems and devices, and iontophoretic (electrical diffusion) delivery systems. Transdermal dosage forms may be used for timed release and sustained release of the active agents of the present invention.

Pharmaceutical compositions and unit dosage forms of the present invention for administration parenterally, and in particular by injection, typically include a pharmaceutically acceptable carrier, as described above. A preferred liquid carrier is vegetable oil. Injection may be, for example, intravenous, intrathecal, intramuscular, intraruminal, intratracheal, or subcutaneous.

The lercanidipine free base can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

In one preferred embodiment, the present invention provides a modified release dosage form comprising a gelatin or hydroxypropylmethylcellulose or other suitable polymers (e.g. exopolysaccharides, such as linear polysaccharides known as polymaltotrioses or pullulans) capsule filled with lercanidipine dissolved or suspended in a Gelucire™ material as described herein, preferably Gelucire™ 50/13 or Gelucire™ 44/14 or a combination a combination thereof. Preferably the ratio of Gelucire™ to lercanidipine is from about 1:500 to about 1:5, more preferably from about 1:250 to about 1:10 still more preferably from about 1:200 to about 1:20. Where the solid oral dosage from comprises more than one Gelucire™ material, the weight ratio of 50/13:44/14 of within the range of from about 1:99 to about 99:1 In forming the modified released lercanidipine pharmaceutical composition of the invention, the lercanidipine is dissolved in a melt of polyglycolized glyceride(s). The mixture in the form of a melt comprising polyglycolized glyceride(s) and lercanidipine and/or other excipients dispersed therein may be filled into hard or soft gelatin or hydroxypropylmethylcellulose or other polymeric component (e.g. pullulans) capsules.

In an additional embodiment, the pharmaceutical composition comprising polyglycolized glyceride and lercanidipine, may be powdered by milling at a low temperature and then incorporated into tablets, beads or beadlets employing conventional procedures. The beads or beadlets may also be formed by the process of prilling where the melt is added dropwise to a non-miscible liquid maintained at a lower temperature.

In yet another embodiment, tablets or pills or granules can be coated or otherwise compounded to form a unit dosage form which has preferably, a modified release profile. For example, the tablet or pill can comprise an inner dosage and an outer dosage component, the latter being in the form of a layer or envelope over the former. The two components can be separated by a release modifying layer which serves to permit dissolution of the active ingredient from the core component over a prolonged period of time. Alternatively, the release modifying agent is a slowly disintegrating matrix. Additional modified release formulations will be apparent to those skilled in the art.

Biodegradable polymers for controlling the release of the active agents, include, but are not limited to, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Administration

The pharmaceutical composition or unit dosage forms of the present invention may be administered by a variety of routes such as intravenous, intratracheal, subcutaneous, oral, mucosal parenteral, buccal, sublingual, ophthalmic, pulmonary, transmucosal, transdermal, and intramuscular. Unit dosage forms also can be administered in intranasal form via topical use of suitable intranasal vehicles, or via transdermal routes, using of transdermal skin patches known to those of ordinary skill in the art. Oral administration is preferred.

The pharmaceutical composition or unit dosage forms of the present invention may be administered to an animal, preferably a human being, in need of antihypertensive treatment. The pharmaceutical composition or unit dosage form of the present invention may be administered according to a dosage and administration regimen defined by routine testing in light of the guidelines given above in order to obtain optimal antihypertensive activity and a decreased in blood pressure while minimizing toxicity or side-effects for a particular patient. However, such fine turning of the therapeutic regimen is routine in light of the guidelines given herein.

The dosage of the composition containing substantially pure lercanidipine free base of the present invention may vary according to a variety of factors such as underlying disease state, the individual's condition, weight, sex and age and the mode of administration. For oral administration, the pharmaceutical compositions can be provided in the form of scored or unscored solid unit dosage forms.

The pharmaceutical composition or unit dosage form may be administered in a single daily dose, or the total daily dosage may be administered in divided doses. In addition, co-administration or sequential administration of other active agents may be desirable. The amorphous form thereof of the invention may be combined with any known drug therapy, preferably for treatment of hypertension. For example, a pharmaceutical composition of the present invention may be combined with an ACE inhibitor, such as enalapril, described in U.S. published application no. 2003/00180355, or with lisinopril as described in commonly-owned U.S. published application no. 2004/0147566. Pharmaceutical compositions comprising substantially pure lercanidipine free base may also be combined with an angiotensin II receptor blocker (ARB), as disclosed in U.S. published application no. 2004/0198789, for example. Also contemplated by the present invention is addition of a diuretic or a receptor blocker to the composition comprising substantially pure lercanidipine free base. Exemplary diuretics include thiazide diuretics, potassium sparing diuretics, loop diuretics, such as hydrochlorothiazide, spironolactone, and ethacrynic acid, respectively.

For combination therapy the compounds may initially be provided as separate dosage forms until an optimum dosage combination and administration regimen is achieved. Therefore, the patient may be titrated to the appropriate dosages for his/her particular hypertensive condition. After the appropriate dosage of each of the compounds is determined to achieve a decrease of the blood pressure without untoward side effects, the patient then may be switched to a single dosage form containing the appropriate dosages of each of the active agents, or may continue with a dual dosage form.

The exact dosage and administration regimen utilizing the combination therapy of the present invention is selected in accordance with a variety of factors including type, species, age, weight, sex and medical condition of the patient; the severity and etiology of the hypertension to be treated; the route of administration; the renal and hepatic function of the patient; the treatment history of the patient; and the responsiveness of the patient. Optimal precision in achieving concentrations of compounds within the range that yields efficacy without toxicity requires a regimen based on the kinetics of the drug's availability to target sites. This involves a consideration of the absorption, distribution, metabolism, excretion of a drug, and responsiveness of the patient to the dosage regimen. However, such fine tuning of the therapeutic regimen is routine in light of the guidelines given herein.

Generally, a dosage form for parenteral administration contains not less then 0.1%, preferably from about 0.5% to about 30%, by weight of substantially pure lercanidipine free base, based upon the total weight of the dosage form. Transdermal dosage forms contain from about 0.01% to about 100% by weight of the active agents, based upon 100% total weight of the dosage.

In a preferred embodiment of the present invention, the composition is administered daily to the patient. In a further preferred embodiment, the pharmaceutical composition or dosage form is administered daily in an amount in the range from about 0.1 to 400 mg of substantially pure lercanidipine free base, more preferably from about 1 to 200 mg, and even more preferably from about 5 to 40 mg.

Preferably upon administration of the substantially pure lercanidipine free base of the present invention, a patient's blood pressure is reduced rapidly by a predetermined increment. Preferably the reduction of systolic blood pressure is in the range from about 20 to about 30 mm Hg, and most preferably about 25 mm Hg, following the administration of 20 mg of substantially pure lercanidipine free base. Preferably the reduction of diastolic blood pressure is in the range from about 10 to about 20 mm Hg, and most preferably about 15 mm Hg, following the administration of 20 mg of substantially pure lercanidipine free base.

### EXAMPLES

The following examples of substantially pure lercanidipine free base, as well as methods of preparing the free base and modified release pharmaceutical compositions comprising the same are now disclosed. The examples are illustrative in nature of the various aspects of the present invention and are not intended to be limiting in any manner.

### Example 1

### Preparation of Substantially Pure Lercanidipine Free Base

(a) Preparation of Free Base from Lercanidipine Hydrochloride in Sodium Borate

A solution of lercanidipine hydrochloride was prepared by mixing 11.3 g of lercanidipine hydrochloride (Recordati S.p.A., Milan, Italy) with 50 mL of methanol (MeOH) at room temperature. A basic medium was prepared by mixing 3.81 g of sodium borate (Na₂B₄O₇.10H₂O) in 1000 mL of water, yielding a basic medium having a pH of 9.2. The lercanidipine hydrochloride solution was added to the basic medium and stirred at room temperature for about ninety minutes. Following stirring the mixture was allowed to stand overnight at room temperature. The resulting yellow solid was separated by filtration using a Buchner funnel. The yellow solid was washed three times with water (200 mL per wash) and then dried at room temperature on P205 under vacuum. The reaction yielded 10.62 g of lercanidipine free base having an HPLC purity of 100% as determined using a Waters 1050 HPLC system (Waters SpA-Vimodrone (Milan) Italy). The chemical composition of the free base prepared by the present example is shown in Table 1, below.

TABLE 1
Chemical Composition of Lercanidipine Free Base

| | % Calculated | % Found | % Corrected¹ |
|---|---|---|---|
| C | 70.68 | 70.19 | 70.42 |
| H | 6.76 | 6.94 | 6.77 |
| N | 6.87 | 6.85 | 6.84 |
| Cl | — | <0.1 | — |
| H₂O | | 0.37 | 0.37 |

¹Calculated values corrected for detected water content.

(b) Preparation of Free Base from Lercanidipine Hydrochloride in Sodium Hydroxide

A solution of lercanidipine hydrochloride was prepared by mixing 11.3 g of lercanidipine hydrochloride (Recordati S.p.A., Milan, Italy) with 50 mL of methanol (MeOH) at room temperature. A basic medium was prepared by diluting sodium hydroxide in water, yielding a 0.1 N solution of sodium hydroxide. The lercanidipine hydrochloride solution was added to 210 mL of the above basic medium and stirred at room temperature for about ninety minutes. Following stirring the mixture was allowed to stand overnight at room temperature. The resulting yellow solid was separated by filtration using a Buchner funnel. The yellow solid was washed three times with water (200 mL per wash) and then dried at room temperature on P₂O₅ under vacuum.

(c) Preparation of Free Base from Lercanidipine Hydrochloride in Potassium Carbonate

A mixture of lercanidipine hydrochloride (750 g), 3000 mL of ethyl acetate and 1800 mL of deionized water was stirred at room temperature. To the solution 479 g of potassium carbonate was added in four portions over 25 minutes. The solution was mixed for 90 minutes, after which, the aqueous phase was discarded and the organic solution washed with 750 mL of deionized water. The organic solution was then evaporated to dryness under vacuum (70° C.-100 mbar) to recover crude lercanidipine free base. The chemical composition of the free base prepared by the present example is shown in Table 2, below.

TABLE 2
Chemical Composition of Lercanidipine Free Base

| | % Calculated | % Found | % Corrected¹ |
|---|---|---|---|
| C | 70.68 | 70.61 | 70.42 |
| H | 6.76 | 6.78 | 6.77 |
| N | 6.87 | 6.82 | 6.84 |
| Cl | — | <0.1 | — |
| H₂O | | 0.36 | 0.36 |

¹Calculated values corrected for detected water content.

(d) Preparation of Free Base from Lercanidipine Hydrochloride with Sodium Methoxide

A solution of lercanidipine hydrochloride was prepared by mixing 700 g of lercanidipine hydrochloride (Recordati SpA, Milan, Italy) with 2800 mL of methanol (MeOH). To this solution there was added, over a period of 30 minutes, 350 mL of 25% sodium methoxide in methanol at room temperature. The resulting methanolic suspension was stirred at 20° C. to 25° C. for 2 hours, and was then added to 18.4 litres of deionized water over a period of 60 minutes under strong stirring. After 2 hours under slower stirring, the resulting solid was separated by filtration using a Buchner funnel and washed three times with 1225 mL of deionized water. The product was dried to constant weight at room temperature under vacuum under a light stream of nitrogen. Yield was 509.4 g (77.1%).

The crude lercanidipine free base was dissolved in 1125 mL of methanol. The resulting solution was allowed to stand for about 2 hours at 5° C. followed by the addition of 7500 mL of cold deionized water under stirring. After further 2 hour stirring the resulting precipitate was collected by suction and dried at room temperature for 96 hours, with further drying under vacuum (5 mbar at 35° C.) for 20 hours. Yield was 605 g (85%), mp 44° C. to 64° C., HPLC purity=99.55%.

### Example 2

### Comparison of Substantially Pure with Crude Lercanidipine Free Base

The following is a comparative example, comparing substantially pure lercanidipine free base prepared as described in example 1 (a to d) with crude free base prepared by the method of U.S. Pat. No. 4,705,797 and lercanidipine hydrochloride. Crude free base was prepared as described below. Lercanidipine hydrochloride was obtained from Recordati S.p.A., Milan, Italy.

Crude lercanidipine free base was prepared by the method of the prior art by cyclizing a solution of 2.37 g of methyl 3-aminocrotonate and 10.29 g of 1,1,N-trimethyl-N-(3,3-diphenylpropyl)-2-aminoethyl α-acetyl-3-nitrocinnamate in 15 mL of isopropanol. The mixture was refluxed for 3 hours. The mixture was then cooled and evaporated to dryness under vacuum. The resulting oily residue was purified by flash chromatography on silica gel columns, using chloroform with increasing amounts of acetone as eluent. The unitary TLC fractions (chloroform: acetone, 9:1 by volume) were evaporated.

The lercanidipine base prepared as described in Example 1 had a greater HPLC purity then the free base prepared by the method of the prior art (see Table 3, below). A complete comparison of physical properties is shown in Table 3.

TABLE 3
Comparison of Physical Properties

| | | Melting Point | Solubility in esters, |
|---|---|---|---|
| | HPLC Purity | (° C.) | ketones, ethers |
| Substantially | 99.5-100% | 44-64 | Very soluble |
| Pure Free Base¹ | | | |
| Lercanidipine | 100% | 186-188 | insoluble |
| Hydrochloride | | | |
| Crude Free Base² | 94% | 38-49 | Very soluble |

¹Prepared as described in Example 1 (a to d)
²Prepared as described in Example 2

### Example 3

### Preparation and Administration of Modified Release Dosage Forms Comprising Substantially Pure Lercan Idipine Free Base to Patients

Different modified release solid unit dosage forms may be prepared as described below. A mixture of lercanidipine free base, Gelucire™, Compritol™ is prepared by first melting the Gelucire™ and Compritol™ by heating to about 90° C. Lercanidipine free base and BHT may be added to the heated mass with continuous mixing until all the added lercanidipine free base has dissolved. Into the melted mass Methocel K4M is dispersed under stirring. The lercanidipine/Gelucire™/Compritol™/Methocel™ mixture is then filled into size #0 hard gelatin capsules. Approximately 500 mg of the lercanidipine/Gelucire™/Comprito™/Methocel™ was added to each capsule. The lercanidipine free base/Gelucire™/Compritol™/Methocel™ filled capsules may be allowed to stand at room temperature to solidify.

Modified lercanidipine dosage forms prepared as described as above such that the dosage forms include 2.5, 5, 10, or 20 mg lercanidipine. The dosage forms comprising 2.5, 5, 10, or 20 mg lercanidipine are administered to patients with mild or moderate hypertension once per day at the same time each day for 28 days. Plasma concentration of lercanidipine is measured 24 h after administration of each dose, prior to administration of any subsequent dose. Blood pressure is monitored daily. It is predicted that the plasma levels of lercanidipine measured 24 hours after administration of each dose and immediately prior to administration of a subsequent will be at least 0.5 ng/mL and also predicted that at the end of 28 days blood pressure will be lowered by at least about 15 mm Hg for systolic pressure and/or by about 10 mm Hg for diastolic pressure.

The present invention is not to be limited in scope by the specific embodiments described herein. Various modifications of the invention in addition to those described herein will become apparent to those skilled in the art from the foregoing description. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

We claim:

1. Lercanidipine free base having a purity of at least 95%.

2. Lercanidipine free base according to claim 1 and having a purity of at least about 99%.

3. Lercanidipine free base according to claim 1 and having a purity of at least about 99.5%.

4. A method for the preparation of lercanidipine free base, the method comprising the steps of:
(a) dissolving a lercanidipine salt in an organic solvent to form a solution;
(b) mixing the solution and an aqueous medium having a pH of from 9 to 14; and
(c) isolating lercanidipine free base.

5. A method according to claim 4 in which the aqueous medium is a basic solution comprising an organic base, an inorganic base or an anionic ion exchange resin.

6. A method according to claim 5 in which the organic base is triethylamine, piperazine, tetramethylethylenediamine, ethylenediamine or 4-dimethylaminopyridine.

7. A method according to claim 5 in which the aqueous medium is an inorganic base selected from the group consisting of sodium hydroxide, potassium hydroxide, lithium hydroxide, borax, sodium carbonate and potassium carbonate.

8. A method according to claim 5 in which the aqueous medium is an anionic exchange resin is one having a quaternary ammonium, tertiary sulphonium, quaternary phosphonium or alkyl pyridinium functional group.

9. A method according to claim 4 in which the organic solvent is a protic polar or an aprotic polar solvent.

10. A method according to claim 9 in which the protic polar solvent is methanol, ethanol, propanol or ethylene glycol.

11. A method according to claim 9 in which the aprotic polar solvent is N,N-dimethylformamide, dimethylsulphoxide or dimethylacetamide.

12. A method for the preparation of lercanidipine free base, the method comprising the steps of:
(a) mixing a lercanidipine salt in a water-immiscible organic solvent and water to form a suspension;
(b) mixing the suspension and an inorganic base;
(c) separating the organic phase from the aqueous phase;
(d) evaporating the solvent from the organic phase to yield lercanidipine free base;
(e) dissolving the lercanidipine free base in a water-miscible organic solvent to form a solution;
(f) precipitating the lercanidipine free base from the solution; and
(g) isolating lercanidipine free base.

13. A method according to claim 12 in which the water-immiscible organic solvent is a hydrocarbon, a halogenated hydrocarbon, an ester or an ether.

14. A method according to claim 12 in which the water-immiscible organic solvent is toluene, dichloromethane, methyl acetate, ethyl acetate, diethyl ether or methyl t-butyl ether.

15. A method according to any one of claims 12 in which the inorganic base is potassium hydroxide, sodium hydroxide, potassium carbonate or sodium carbonate.

16. A method according to claim 16 in which the protic polar solvent is methanol, ethanol, propanol or ethylene glycol.

17. A method according to claim 16 in which the aprotic polar solvent is N,N-dimethylformamide, dimethylsulphoxide or dimethylacetamide.

18. A method for the preparation of lercanidipine free base, the method comprising the steps of:
(a) dissolving a lercanidipine salt in a first organic solvent to form a solution;
(b) mixing the solution from step (a) with a solution of a base in a second organic solvent;
(c) mixing the solution from step (b) with water; and
(d) isolating lercanidipine free base.

19. A method according to claim 18 in which the first organic solvent is selected from methanol, ethanol, propanol, ethylene glycol, N,N-dimethylformamide, dimethylsulphoxide, and dimethylacetamide.

20. A method according to claim 18 in which the second organic solvent is selected from methanol, ethanol, propanol, ethylene glycol, N,N-dimethylformamide, dimethylsulphoxide, and dimethylacetamid.

21. A method according to claim 18 in which the base is sodium methoxide, potassium methoxide, lithium methoxide, aluminum methoxide, palladium methoxide, and titanium methoxide.

22. A method according to claim 18 in which the lercanidipine salt is an acid addition salt, wherein the acid counterion is selected from an inorganic acid, a sulphonic acid, a monocarboxylic acid, a dicarboxylic acid, a tricarboxylic acid and an aromatic sulphonimide.

23. A method according to claim 22 in which the lercanidipine salt is an acid addition salt, wherein the acid counterion is selected from hydrochloric acid, hydrobromic acid, phosphoric acid, sulphuric acid, methanesulphonic acid, benzenesulphonic acid, toluenesulphonic acid, napthalene-1,5-disulphonic acid, acetic acid, (+)-L-lactic acid, DL-lactic acid, DL-mandelic acid, gluconic acid, cinnamic acid, salicylic acid, gentisic acid, oxalic acid, 2-oxo-glutaric acid, malonic acid, (−)-L-malic acid, mucic acid, (+)-L-tartaric acid, fumaric acid, maleic acid, terephthalic acid, citric acid and saccharin.

24. A method according to claim 22 in which the lercanidipine salt is lercanidipine hydrochloride.

25. A pharmaceutical composition comprising the lercanidipine free base according to claim 1 and a pharmaceutically acceptable diluent, carrier and/or excipient.

26. A pharmaceutical composition according to claim 25, the composition containing at least one component selected from a pharmaceutically acceptable diluent, flavourant, sweetener, preservative, dye, binder, suspending agent, viscosity increasing agent, dispersing agent, colourant, disintegrant, lubricant, antioxidant, plasticizer and edible oil.

27. A pharmaceutical composition according to claim 26, the composition being adapted for modified release and containing at least one waxy substance.

28. A pharmaceutical composition according to claim 27 in which the waxy substance is a polyalcohol fatty acyl ester or a mixture of polyalcohol fatty acyl esters.

29. A pharmaceutical composition according to claim 28 in which the or each polyalcohol fatty acyl ester is a polyethylene glycol ester, a polypropylene glycol ester or a fatty acid glyceride.

30. A pharmaceutical composition according to claim 28 in which the waxy substance is a polyglycolized glyceride comprising a fatty acid ester and a polyethylene glycol ester, the polyglycolized glyceride having a melting of point from 33° C. to 64° C. and an HLB value from 1 to 14.

31. A pharmaceutical composition according to claim 27, the composition being in unit dose form, and being contained in a gelatin, hydroxypropylmethylcellulose or pullulans capsule.
